(19) United States
(12) Patent Application Publication
(10) Pub. No.: US 2025/0109080 A1
(43) Pub. Date: Apr. 3, 2025

(54) CONVERSION OF METHANOL TO A HYDROCARBON PRODUCT STREAM

(71) Applicant: Topsoe A/S, Kgs. Lyngby (DK)

(72) Inventors: Sudip DE SARKAR, KOKKEDAL (DK); Christian WIX, Nærum (DK); Kim AASBERG-PETERSEN, Allerød (DK)

(73) Assignee: Topsoe A/S, Kgs. Lyngby (DK)

(21) Appl. No.: 18/834,426

(22) PCT Filed: Mar. 13, 2023
(86) PCT No.: PCT/EP2023/056334

§ 371 Date: Jul. 30, 2024

(30) Foreign Application Priority Data
Mar. 14, 2022 (EP) 22161938.0

(51) Int. Cl. C07C 1/04

(52) U.S. Cl. CPC C07C 1/041; C07C 1/0495

(57) ABSTRACT

The present invention relates to a hydrocarbon plant and related process, in which a first feed of methanol, having a water content of less than 10% by weight, is converted in a syngas generation stage (A) into at least a syngas stream. A synthesis stage (B) is arranged to receive the syngas stream and convert it to at least a hydrocarbon product stream and an off-gas stream. The plant and process of the present invention use better-understood process steps compared to known processes/plants and provide the opportunity for a reduction in CO₂ emissions.

### TECHNICAL FIELD

The present invention relates to a hydrocarbon plant, and a process for producing a hydrocarbon product stream in such a plant from methanol feed. The plant and process of the present invention provide the opportunity for a reduction in CO₂ emissions compared to known processes/plants.

### BACKGROUND

Methanol is the simplest alcohol and is the building block of myriad of useful chemical products, used in everyday life. These chemical products include gasoline, olefins, formaldehyde and many more. Production of methanol from both conventional fossil resources and sustainable feeds (such as CO₂ and H₂, biomass etc.) are well-established in the process industry. With increasing focus on carbon capture and utilization (CCU), there is a burgeoning interest in producing these useful chemical products from sustainable feeds via methanol.

However, production of a set of hydrocarbon fuels, such as—diesel and/or jet-fuel and/or kerosene etc., from methanol is not well-known. Traditionally, these diesel and/or jet-fuel and/or kerosene products can be produced from synthesis gas, obtained from either steam methane reforming of conventional fossil-based resources or reverse water shift of CO₂+H₂ feed or gasification of biomass feed, via the Fischer-Tropsch (F-T) and downstream product work-up.

Research is ongoing for converting methanol to diesel, jet-fuel etc. For example, feeding methanol to Fischer-Tropsch process is still unknown. The invention, described in this document, provides a method for production of diesel, jet-fuel etc. from a methanol feed. In current invention, methanol feed is cracked in presence of catalysts to produce synthesis gas stream and then the said synthesis gas stream is converted to diesel and/or jet-fuel and/or kerosene etc. via the Fischer-Tropsch (F-T) and downstream product work-up, as per traditional process. This process and plant are specifically interesting when both methanol and diesel, jet-fuel, kerosene etc. are required as products. Alternatively, the current invention becomes relevant when at least a part of methanol from existing system needs to be converted to diesel, jet-fuel, kerosene etc.

Related patent publications on methanol cracking include WO2021063794 and WO2021063796.

### SUMMARY

One method for the production of a hydrocarbon product stream could be via direct feed of methanol to an F-T section. However, the impact of this arrangement on F-T process is unknown. Therefore, an alternative process is required in which methanol production can be controlled and hydrocarbon fuels, such as—diesel and/or jet-fuel and/or kerosene etc. can be made from the said methanol stream. It is also advantageous to be able to provide two high-grade product streams—methanol stream and hydrocarbon product stream—in a single plant/process.

The present technology provides a hydrocarbon plant according to independent claim 1, and a process according to independent claim 16.

The present technology allows the production of a methanol product stream and a hydrocarbon product stream (which can be upgraded to a hydrocarbon fuel stream). Additionally, the endothermic MeOH cracking process requires energy input which can be provided via an e-reactor, thus reducing CO₂ emissions, if the electricity source is sustainable.

Further details of the technology are provided in the enclosed dependent claims, figures and examples.

### LEGENDS TO THE FIGURES

The technology is illustrated by means of the following schematic illustrations, in which:

FIG. 1 shows a first layout of the plant/process of the invention.

FIG. 2 shows a second layout of the plant/process of the invention.

FIG. 3 shows a third layout of the plant/process of the invention.

FIG. 4 shows a fourth layout of the plant/process of the invention.

FIG. 5 shows a fifth layout of the plant/process of the invention.

FIG. 6 shows a sixth layout of the plant/process of the invention.

FIG. 7 is a graph showing changes in H₂/CO ratio, CO₂ and CH₄ composition of a syngas with changes in water content in a MeOH feed.

### DETAILED DISCLOSURE

Unless otherwise specified, any given percentages for gas content are % by volume. All feeds are preheated as required.

The term “synthesis gas” (abbreviated to “syngas”) is meant to denote a gas comprising hydrogen, carbon monoxide, carbon dioxide and small amounts of other gasses, such as argon, nitrogen, methane, etc.

The term “hydrocarbon fuel” includes diesel, jet fuel, kerosene, e.g. synthetic paraffinic kerosene, LPG and/or naphtha. Of these, jet-fuel is preferred.

As noted above, a hydrocarbon plant is provided. In general terms, the plant comprises:
a first feed of methanol,
a syngas generation stage (A) comprising at least a methanol cracking unit,
optionally, a hydrogen separation stage (A1), and
a synthesis stage (B).

Details of the components which make up the plant are described in the following.

A first feed of methanol is supplied to the plant. This first feed of methanol has a water content of less than 10% by weight, preferably less than 5% by weight, more preferably less than 2% by weight. The first feed of methanol can be sourced from any conceivable methanol synthesis process. This may be, for example, a process involving conversion of H₂ and CO₂ to MeOH, syngas to methanol, bio-mass gasification to MeOH or biogas conversion to MeOH.

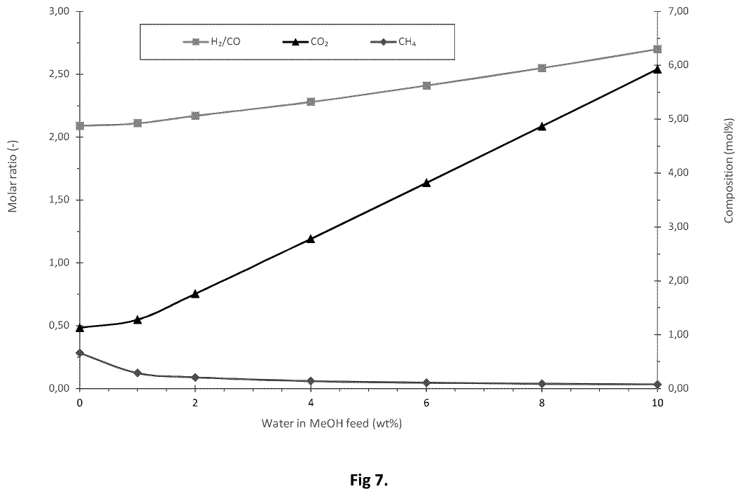

It is found advantageous to perform MeOH cracking with a low water content (see FIG. 7 and accompanying explanation). At the same time, the complete absence of water in the methanol feed provides a slow methanol cracking reaction rate, and requires higher catalyst volume. Preferably, therefore the first feed of methanol, has a water content of at least 0.1% by weight, preferably more than 0.5% by weight, more preferably more than 1% by weight. The first feed of methanol may comprise small amounts of other substances, such as ethanol, propanol and other alcohols, which are typical impurities in fuel grade methanol.

The plant comprises syngas generation stage (A), as set out above. The function of this stage is to provide a syngas stream by cracking the first feed of methanol. Accordingly, the syngas generation stage comprises at least a methanol cracking unit, said methanol cracking unit being arranged to receive the first feed of methanol and convert it into at least a first syngas stream. The said first syngas is cooled and optionally, unconverted methanol is separated from said cooled first syngas by using separator, optionally provided with washing trays or packed bed column, to provide a second syngas stream and a condensate stream, which may comprise unconverted methanol. The said condensate stream can optionally be recycled and mixed to the main methanol feed. Alternatively, the said condensate stream can be fed to upstream water removal section, where at least a part of water is removed from raw methanol feed. Additionally, the syngas generation stage (A) may comprise at least a methanol feed evaporation unit before feeding it to methanol cracking unit.

Methanol cracking can take place by one or more of three reactions:
1. CH₃OH↔2H₂+CO (dry MeOH cracking)
2. CO+H₂O↔H₂+CO₂ (water gas shift)
3. CH₃OH+H₂O↔3H₂+CO₂ (wet MeOH cracking)

Reaction 1 is preferred, so that the syngas exiting the methanol cracking unit has a H₂:CO ratio closer to the required value for the downstream F-T section (ca. 2.00)—see FIG. 7 and related discussion.

In the presence of water, reaction 2 is promoted, which converts CO to CO₂ and H₂, causing increase in H₂:CO ratio. If MeOH cracking is performed in presence of sufficient amount of steam (i.e., reaction 3—wet MeOH cracking), virtually all CO can be converted to CO₂ and H₂, thus maximizing H₂ production. Therefore, the plant/process including methanol cracking with limited water content has the potential to provide a first syngas stream from the cracking unit with a low content of CO₂.

Furthermore, the wet MeOH cracking reaction is faster than the dry methanol cracking reaction. Therefore, a small amount of water (less than 10% by weight, preferably less than 5% by weight, more preferably less than 2% by weight) in the MeOH feed would be advantageous.

Additionally, the plant/process including methanol cracking has the potential to provide a syngas stream to the downstream process (e.g. FT synthesis) which prefers low content of CO₂, methane and other alkanes.

The first synthesis gas stream produced by the methanol cracking unit suitably has the following content of gaseous components:
40-70% H₂ (dry)
10-35% CO (dry)
1-10% CO₂ (dry)
0.1-1% CH₄

The methanol cracking unit may be any type of methanol cracking unit, including a fired methanol cracking unit and an electrically heated methanol cracking unit. In a particularly preferred aspect, the methanol cracking unit is an electrical methanol (eMeOH) cracking unit, e.g. heated by induction heating or resistance heating. A particular embodiment of the electrical methanol (eMeOH) cracking unit comprises:
a structured catalyst arranged for catalyzing the methanol cracking reaction of said methanol stream, said structured catalyst comprising a macroscopic structure of electrically conductive material, said macroscopic structure supporting a ceramic coating, wherein said ceramic coating supports a catalytically active material;
a pressure shell housing said structured catalyst, said pressure shell comprising an in-let for letting in said methanol stream and an outlet for letting out syngas, wherein said inlet is positioned so that said methanol stream enters said structured catalyst in a first end of said structured catalyst and the first syngas stream exits said structured catalyst from a second end of said structured catalyst;
a heat insulation layer between said structured catalyst and said pressure shell;
at least two conductors electrically connected to said structured catalyst and to an electrical power supply placed outside said pressure shell, wherein said electrical power supply is dimensioned to heat at least part of said structured catalyst to a temperature of at least 150° C. by passing an electrical current through said macroscopic structure, preferably wherein said at least two conductors are connected to the structured catalyst at a position on the structured catalyst closer to said first end of said structured catalyst than to said second end of said structured catalyst, and wherein the structured catalyst is constructed to direct an electrical current to run from one conductor substantially to the second end of the structured catalyst and return to a second of said at least two conductors;
an outlet for the first syngas stream.

The layout of the reactor system allows for feeding a pressurized methanol stream to the reactor system at an inlet and directing this gas into the pressure shell of the reactor system. Inside the pressure shell, a configuration of heat insulation layers and inert material is arranged to direct the methanol stream through the structured catalyst where it will be in contact with the catalyst material, where the catalytically active material will facilitate the methanol cracking reaction. Additionally, the heating of the structured catalyst will supply the required heat for the endothermic reaction. The first syngas stream from the heated structured catalyst is led to the reactor system outlet.

The close proximity between the catalytically active material and the electrically conductive materials enables efficient heating of the catalytically active material by close proximity heat conduction from the resistance heated electrically conductive material. An important feature of the resistance heating process is thus that the energy is supplied inside the object itself, instead of being supplied from an external heat source via heat conduction, convection and radiation. Moreover, the hottest part of the reactor system will be within the pressure shell of the reactor system. Preferably, the electrical power supply and the structured catalyst are dimensioned so that at least part of the structured catalyst reaches a temperature of at least 150° C., preferably at least 300° C. The surface area of the electrically conductive material, the fraction of the electrically conductive material coated with a ceramic coating, the type and structure of the ceramic coating, and the amount and composition of the catalytically active catalyst material may be tailored to the specific reaction at the given operating conditions.

Syngas obtained from MeOH cracking, as described herein, has much less CO₂ than conventional syngas stream from alternative routes. This provides a margin to add some CO₂ feed to decrease the H₂/CO ratio without increasing CO₂ content beyond what is usually seen in syngas for FT synthesis. Because of low CO₂ content in the syngas from MeOH cracking, addition of CO₂ to the methanol cracking unit wouldn't affect CO₂ content in syngas too adversely. In one aspect, the plant therefore comprises a CO₂-rich feed to the methanol cracking unit.

Further details of the electrical methanol (eMeOH) cracking unit are provided in WO 2021/063794, the contents of which are hereby incorporated by reference.

In addition to the MeOH cracking unit, stage (A) may comprise one or more heat exchangers.

In a preferred embodiment, the stoichiometry of H₂ and CO in the first syngas stream falls within an interval such that the first syngas stream has a H₂/CO ratio of between 1.8 and 2.2, preferably between 1.9 and 2.1.

In one embodiment, the system may further comprise a hydrogen separation stage (A1). This separation stage is arranged to receive at least a portion of the first or second syngas stream and provide a third syngas stream and a hydrogen-rich stream. The third syngas stream thus has a lower hydrogen content than the first and second syngas stream. The hydrogen separation stage (A1) can be used to obtain a H₂/CO ratio which is suitable for downstream FT synthesis, preferably between 1.8 to 2.2, more preferably between 1.9 to 2.10. The syngas stream at the inlet of the Fischer-Tropsch (F-T) section (which is suitably the third syngas stream in the current embodiment) has a hydrogen/carbon monoxide ratio in the range 1.00-3.00; preferably 1.50-2.0, more preferably 1.50-2.10.

At the same time, a hydrogen rich stream is produced. The hydrogen rich stream can be used in methanol production Alternatively, hydrogen rich stream can be used in the product workup unit (PWU) downstream the synthesis stage. The hydrogen separation stage A1 may comprise a membrane hydrogen separation unit or a PSA (pressure swing adsorption) unit or both.

A synthesis stage B is arranged to receive the first or second syngas stream from the syngas generation stage (A) and convert it to at least a hydrocarbon product stream and an off-gas stream. In one embodiment, the synthesis stage B is arranged to receive at least a portion of the first or the second syngas stream and/or—where hydrogen separation stage (A1) is present—at least a portion of the third syngas stream and convert it to at least a hydrocarbon product stream and an off-gas stream.

### Fischer-Tropsch (F-T) Section

The synthesis stage (B) preferably comprises a Fischer-Tropsch (F-T) synthesis unit. Fischer-Tropsch technology is well-established, and typically provides hydrocarbon product stream from a syngas stream. The hydrocarbon product stream can subsequently be worked-up to form a hydrocarbon fuel stream (such as jet fuel, kerosene, diesel and/or naphtha).

At least a portion of the first or second and/or third syngas streams is supplied to a Fischer-Tropsch (F-T) section and converted therein into at least a hydrocarbon product stream and an F-T off-gas stream. The ratio between long chain hydrocarbons and olefins in the hydrocarbon product from the F-T section depends on the type of catalyst, reaction temperature etc. that are used in the process.

An F-T off gas stream is produced as side product. The F-T off gas stream typically comprises carbon monoxide (10-40 vol. %), hydrogen (10-40 vol %), carbon dioxide (10-50 vol %), and methane (10-40 vol %). Additional components such as argon, nitrogen, olefins, and paraffins with two or more carbon atoms may also be present in smaller amounts.

### Product Work-Up Unit (PWU)

The product workup unit (PWU) is arranged to receive at least a portion of the hydrocarbon product stream and provide a hydrocarbon fuel product stream. Suitably, the hydrocarbon fuel stream is a jet fuel stream, a diesel stream, a naphtha stream, an LPG stream and/or a kerosene stream. The product workup unit PWU is alternatively known as an “upgrading unit”.

In one embodiment, the hydrocarbon fuel product stream comprises primarily C12-C15 hydrocarbons. The PWU may also provide one or more off-gas streams, which-depending on the composition of the hydrocarbon product stream—may comprise e.g. lower (<C10 hydrocarbons).

Syngas generated from a parallel process can be mixed with second syngas from syngas generation stage (A) comprising methanol cracking unit, to obtain a mixed syngas for downstream FT synthesis. Therefore, the plant may further comprise an off-gas reforming stage (B1). This reforming stage (B1) is arranged to receive at least a portion of the off-gas stream from the synthesis stage (B), and convert it to a fourth syngas stream. At least a portion of said fourth syngas stream is arranged to be fed to the inlet of the synthesis stage (B), preferably in admixture with said at least a portion of the first or second syngas stream and/or—where present—said at least a portion of the third syngas stream. In one embodiment, the off-gas reforming stage (B1) is an electrical reforming section, preferably an electrical steam methane reforming (eSMR) reactor and/or an electrical reverse water gas shift (eRWGS) reactor.

In this embodiment, preferably the stoichiometry of H₂ and CO in the combined syngas stream fed into the synthesis stage (B) comprising the first or second syngas stream and the fourth syngas stream, and/or the third syngas stream, if present, falls within an interval such that the first syngas stream has a H₂/CO ratio of between 1.8 and 2.2, preferably between 1.9 and 2.1.

In a particular embodiment, the H₂/CO ratio of the first or second syngas stream is higher than 2.0, and the H₂/CO ratio of the fourth syngas stream and/or the third syngas stream, if present, is lower than 2.0. In this embodiment, preferably the stoichiometry of H₂ and CO in the combined syngas stream fed into the synthesis stage (B) comprising the first second syngas stream and the fourth syngas stream, and/or the third syngas stream, if present, falls within an interval such that the first syngas stream has a H₂/CO ratio of between 1.8 and 2.2, preferably between 1.9 and 2.1.

The off-gas from the synthesis stage (B) may be sent to an off-gas treatment stage (A2). The off-gas treatment stage (A2) is arranged to receive at least a portion of the off-gas stream from the synthesis stage (B), and provide a treated off-gas. The off-gas treatment stage (A2) comprises at least one hydrogenation unit to hydrogenate olefins, present in the off-gas. Furthermore, off-gas treatment stage (A2) may comprise at least one waste gas shift (WGS) reactor, optionally followed by at least one pre-converter to convert all higher hydrocarbons to lower hydrocarbons, mainly methane. At least a portion of said treated off-gas may be fed to the methanol cracking unit in syngas generation stage (A) and/or to the off-gas reforming stage (B1). Optionally, the first syngas from methanol cracking unit comes out at a high temperature preferably more than 700° C., more preferably more than 800° C., more preferably more than 900° C. and even more preferably more than 1000° C.

The plant described herein is particularly useful when combined with an upstream methanol synthesis stage, designated M. In one aspect, the plant comprises a methanol synthesis stage (M) comprising at least one methanol synthesis unit, and hydrogen and carbon dioxide feeds to the methanol synthesis stage (M).

A hydrogen feed is provided to the methanol synthesis stage (M). Suitably, the hydrogen feed consists essentially of hydrogen. The hydrogen feed is suitably “hydrogen rich” meaning that the major portion of this feed is hydrogen, i.e. over 75%, such as over 85%, preferably over 90%, more preferably over 95%, even more preferably over 99% of this feed is hydrogen. One source of the hydrogen feed can be one or more electrolyser units. In addition to hydrogen the hydrogen feed may for example comprise steam, nitrogen, argon, carbon monoxide, carbon dioxide, and/or hydrocarbons. In some cases, a minor content of oxygen may be present in this hydrogen feed, typically less than 100 ppm. The hydrogen feed suitably comprises only low amounts of hydrocarbon, such as for example less than 5% hydrocarbons or less than 3% hydrocarbons or less than 1% hydrocarbons.

A carbon dioxide feed is provided to the methanol synthesis stage (M). The carbon dioxide feed suitably comprises more than 90% CO₂, preferably more than 95% CO₂, preferably more than 99% CO₂. The carbon dioxide feed may in addition to CO₂ comprise minor amounts of, for example, steam, oxygen, nitrogen, oxygenates, amines, ammonia, carbon monoxide, and/or hydrocarbons.

The carbon dioxide feed suitably comprises only low amounts of hydrocarbon, such as for example less than 5% hydrocarbons or less than 3% hydrocarbons or less than 1% hydrocarbons.

The hydrogen feed and the carbon dioxide feed are—in one aspect—combined prior to being fed to the methanol synthesis stage (M).

The methanol synthesis stage (M) is arranged to convert the hydrogen feed and the carbon dioxide feed into at least a raw methanol stream and a purge stream. Methanol synthesis units suitable for this process are known in the art.

The (raw) methanol stream produced by the methanol synthesis unit suitably comprises more than 40% methanol, preferably more than 50% methanol. The rest of this stream is primarily water. The methanol product stream at this point is usually in liquid form.

A water removal stage (M1) is arranged to receive at least a portion of the raw methanol stream and to provide a methanol stream having a water content of less than 10% by weight, preferably less than 5% by weight, more preferably less than 2% by weight and a water-containing stream. The water removal stage (M1) may suitably comprise one or more distillation units (e.g. distillation columns). A skilled person can identify components of the water removal section which will provide the required separation of streams.

The water removal section therefore provides a “dry” methanol product stream. By the term “dry” is meant that this stream contains less than 10% by weight, such as less than less than 5%, preferably less than 2%, by weight water content.

Complete removal of water from raw methanol might not be required, because methanol cracking with small amount of water can produce a second syngas stream with H₂/CO>2.0. Reforming of F-T off-gas, comprising 20-50 mol % CO₂, will produce a fourth syngas stream with H₂/CO<2.0. Mixing the said second and fourth syngas would result in the final syngas stream with a H₂/CO ratio suitable for synthesis stage comprising an F-T synthesis unit.

The water-containing stream may be used as process water or process steam in the plant.

At least a portion of the methanol stream from the water removal stage (M1) may be provided to the syngas generation stage (A) as the first feed of methanol, specified above.

In one aspect, the syngas generation stage (A) comprising methanol cracking unit is arranged to receive a first portion of the methanol stream from the water removal stage (M1), and a second portion of the methanol stream is provided from the plant (i.e. outputted), if necessary after further purification to a desired quality, as a methanol product stream. Purification to a desired quality typically includes removal of any water which might be present in the second portion of the methanol stream. The plant may further comprise a methanol purification unit, arranged to receive the second portion of the methanol stream from the water removal stage, and provide a purified methanol product stream. The plant according to this embodiment, therefore, allows a methanol product stream and a hydrocarbon product stream to be produced in parallel.

As an alternative, all water can be removed from the raw methanol stream in water removal stage M1 and water subsequently added, so that the desired water content in the first feed of methanol can be achieved.

The present invention also provides a process for providing a hydrocarbon product stream in a hydrocarbon plant as described herein. The process comprises the general steps of:
providing a hydrocarbon plant as described herein,
supplying a first feed of methanol, having a water content of less than 10% by weight, to the syngas generation stage (A) comprising methanol cracking unit,
converting first feed of methanol in the syngas generation stage (A) comprising methanol cracking unit into at least a first syngas stream, which is optionally cooled to remove unconverted methanol to form a second syngas stream (300);
optionally, supplying at least a portion of the first or second syngas stream to a hydrogen separation stage (A1), to provide a third syngas stream and a hydrogen-rich stream (260);
supplying the first or second syngas stream and/or the third syngas stream, if present, to the synthesis stage (B) from the syngas generation stage (A), and converting it to at least a hydrocarbon product stream and an off-gas stream.

Suitably, the first feed of methanol has a water content of less than 5%, preferably less than 2%, by weight, for the reasons provided above. Also, the first feed of methanol may have a water content of at least 0.1%, preferably more than 0.5%, more preferably more than 1% by weight.

In an additional embodiment of the process, the plant comprises:
a methanol synthesis stage (M) comprising at least one methanol synthesis unit,
a hydrogen feed to said methanol synthesis stage (M),
a carbon dioxide feed to said methanol synthesis stage (M), and
water removal stage (M1), wherein said process comprises the steps of:
converting the hydrogen feed and the carbon dioxide feeds in said methanol synthesis stage (M) into at least a raw methanol stream and a purge stream,
supplying at least a portion of the raw methanol stream from methanol synthesis stage (M) to the water removal stage (M1), to provide a methanol stream having a water content of less than 10% by weight, and a water-containing stream,
supplying at least a portion of the methanol stream from said water removal stage (M1) to said syngas generation stage (A) comprising at least one at least one methanol cracking unit as the first feed of methanol.

In this embodiment, the syngas generation stage (A) comprising at least one methanol cracking unit may be arranged to receive a first portion of the methanol stream from the water removal stage (M1), and wherein a second portion of the methanol stream is provided from the plant as a methanol product stream. If necessary, methanol product stream can be passed through further purification steps in a methanol purification stage to meet desired quality for intended use of methanol product. The process according to this embodiment, therefore, allows a methanol product stream and a hydrocarbon product stream to be produced in parallel.

In one particular aspect, in which the syngas generation stage (A) comprises a hydrogen separation stage (A1), being arranged to receive at least a portion of the first or second syngas stream and provide a third syngas stream and a hydrogen-rich stream, the process may comprise the step of supplying at least a portion of the first or second syngas stream and/or at least a portion of the third syngas stream to the synthesis stage (B), and converting it to at least a hydrocarbon product stream and an off-gas stream.

### Specific Embodiments

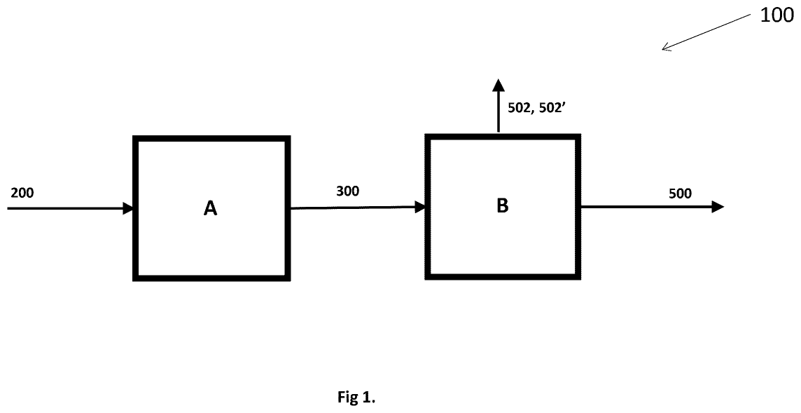

FIG. 1 shows a first layout of the plant used in the process of the invention. A first feed 200 of methanol, having a water content of less than 10% by weight, is fed to a syngas generation stage (A) comprising at least one methanol cracking unit. The said syngas generation stage (A) comprising at least one methanol cracking unit receives the first feed 200 of methanol and convert it into at least a second syngas stream 300.

At least a portion of the first syngas stream 300 is fed to synthesis stage (B), where it is converted hydrocarbon product stream 500 and an off-gas stream 502, 502′.

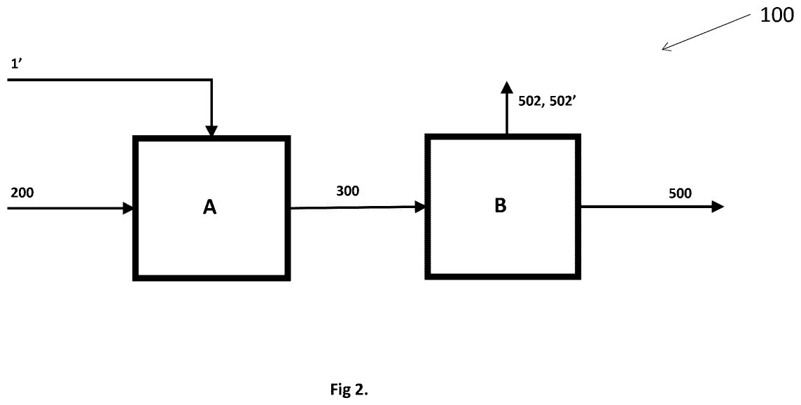

FIG. 2 shows a layout similar to FIG. 1, additionally comprising a CO₂ feed 1′ to the syngas generation stage (A) comprising at least one methanol cracking unit. Feeding a CO₂-feed, if needed after preheating, to the at least one methanol cracking unit in the said syngas generation stage (A) enhances the reverse water gas shift (RWGS) reaction, i.e. the reverse of the reaction 2 listed above.

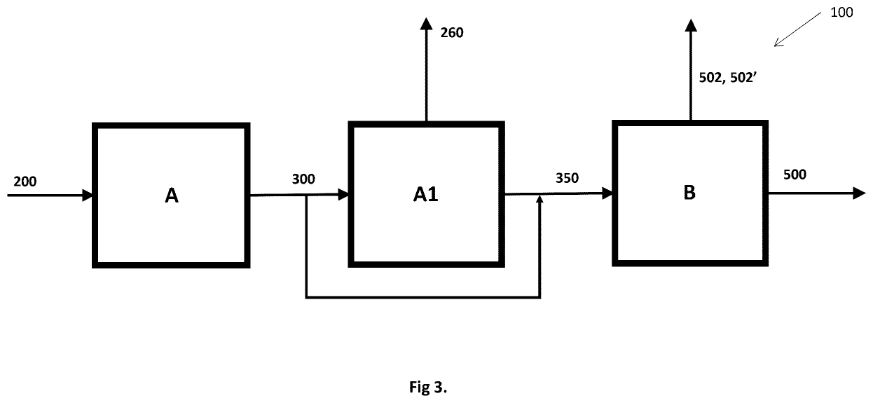

FIG. 3 shows a layout similar to FIG. 1, additionally comprising a hydrogen separation stage (A1). A portion of the second syngas stream 300 is passed through this hydrogen separation stage (A1), and a third syngas stream 350 is provided, having a lower hydrogen content than the second syngas stream 300. A hydrogen-rich stream 260 is provided at the same time. In the particular embodiment of FIG. 3, a portion of the second syngas stream 300 bypasses the hydrogen separation stage A1, and is combined with the third syngas stream 350 before the mixed syngas stream is fed to the synthesis stage (B).

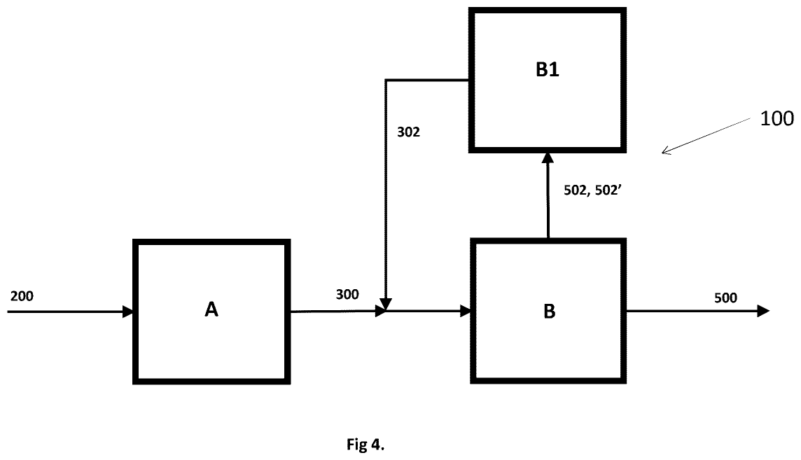

FIG. 4 shows a layout similar to that of FIG. 1. The layout of FIG. 4 includes an off-gas reforming stage (B1) which is arranged to receive the off-gas stream 502, 502′ from the synthesis stage (B). This stream is converted in the off-gas reforming stage (B1) to a fourth syngas stream 302. As shown, the fourth syngas stream 302 is fed to the inlet of the synthesis stage (B)—in this case—in admixture with the second syngas stream 300.

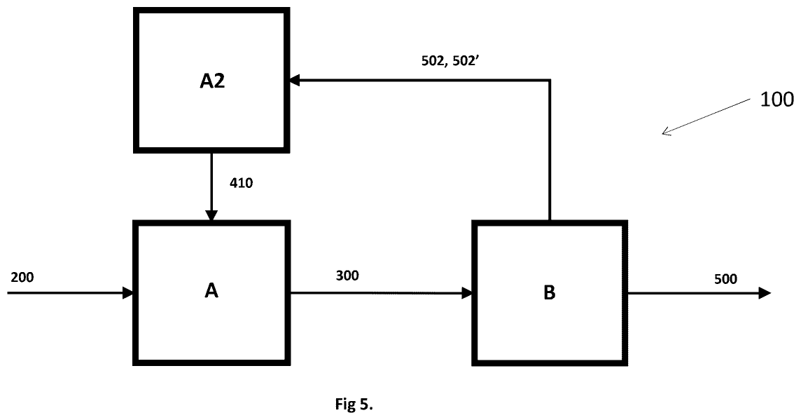

FIG. 5 shows a layout similar to that of FIG. 1. The layout of FIG. 5 includes an off-gas treatment stage (A2). The off-gas treatment stage (A2) is arranged to receive the off-gas stream 502 from the synthesis stage (B), and provide a treated off-gas 410. The treated off-gas 410 is then fed to syngas generation stage (A) comprising at least one methanol cracking unit.

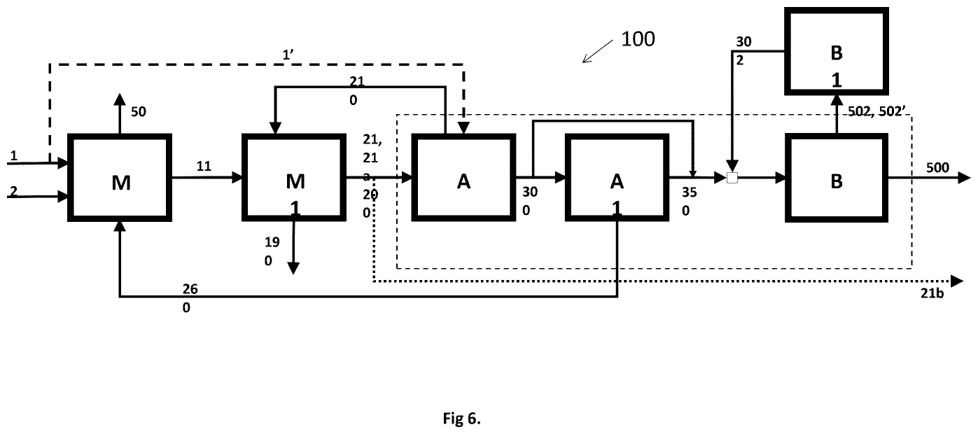

FIG. 6 shows a layout, having a plant layout which is a combination of the layouts of FIGS. 3 and 4. Additionally, the layout of FIG. 6 shows methanol synthesis stage (M) comprising at least one methanol synthesis unit. Plant feeds in FIG. 6 are carbon dioxide feed 1 and hydrogen feed 2.

In FIG. 6, the carbon dioxide feed 1, and the hydrogen feed 2 are supplied to the methanol synthesis stage (M), which converts them to a raw methanol stream 11 and purge stream 50. At least a part of the water in raw methanol stream 11 is then removed in the water removal section M1 (which may comprise one or more distillation columns) to provide a methanol stream 21, suitable for cracking, and water-containing stream 190.

A portion of the methanol stream 21a is sent to syngas generation stage (A) comprising at least one methanol cracking unit—as first feed 200 of methanol. A second portion of the methanol stream 21b is optionally outputted as a methanol product stream. The methanol product stream can optionally be further purified depending on the desired use (not shown in the figure).

In syngas generation stage A, the first feed of methanol 200 is converted into a first syngas stream at methanol cracking unit. The said first syngas is then cooled and unconverted methanol feed is separated to provide second syngas stream 300. Hydrogen separation stage (A1) receives the second syngas stream 300 and provide a third syngas stream 350. At the same time, a hydrogen-rich stream 260 is provided, which is fed to methanol synthesis stage (M). Process condensate, potentially comprising unconverted methanol, 210 from the syngas generation stage (A) may be returned to water removal stage (M1) to enhance overall efficiency of the process.

Synthesis stage (B) receives a mix of second and third syngas streams 300, 350 and converts this into a hydrocarbon product stream 500 and an off-gas stream 502, 502′.

Off-gas stream 502, 502′ from the synthesis stage (B) is fed to off-gas reforming stage (B1). This stream is converted in the off-gas reforming stage (B1) to fourth syngas stream 302, which—as per FIG. 4—is fed to the inlet of the synthesis stage (B) in admixture with the second syngas stream 300.

### Examples

Dry cracking can theoretically provide syngas with H₂/CO of 2.0. However, in reality the reaction is accompanied by other side reactions which are more evident in absence of water. Moreover, dry MeOH cracking reaction rate is slow, requiring higher catalyst volume.

The reaction rate can be improved by small amount adding water. Additionally, by-product formation can also be minimized by adding water. Therefore, it is found advantageous to perform methanol cracking with minor water content, such as <10 wt % water, preferably <5 wt %, more preferably <2 wt % water.

FIG. 7 shows changes in H₂/CO ratio, CO₂ and CH₄ composition in syngas with changes in water content in MeOH feed. Low CH₄ and CO₂ content, which is considered as inert in low temperature F-T synthesis, is an advantage for downstream synthesis stage (B) comprising F-T synthesis units. The graph indicates that both CO₂ content, and H₂/CO ratio increase with increase in methanol feed water content. However, at lower water concentration both H₂/CO and CO₂ content is low enough to provide a syngas, suitable for FT synthesis.

The present invention has been described with reference to a number of embodiments and figures. However, the skilled person is able to select and combine various embodiments within the scope of the invention, which is defined by the appended claims. All documents referenced herein are incorporated by reference.

1. A hydrocarbon plant, said plant comprising:
a first feed of methanol, having a water content of less than 10% by weight,
a syngas generation stage comprising at least a methanol cracking unit, said methanol cracking unit being arranged to receive the first feed of methanol and convert it into at least a first syngas stream, and optionally a separator for removing unconverted methanol to form a second syngas stream;
a synthesis stage, being arranged to receive the first or second syngas stream from the syngas generation stage and convert it to at least a hydrocarbon product stream and an off-gas stream.

2. The plant according to claim 1, wherein the first feed of methanol, has a water content of less than 5% by weight.

3. The plant according to claim 1, wherein said synthesis stage comprises at least one Fischer-Tropsch synthesis unit, being arranged to receive the first or second syngas stream from the syngas generation stage, and convert it to at least a hydrocarbon product stream and an F-T off-gas stream.

4. The plant according to claim 3, wherein further comprising a product workup unit arranged to receive at least a portion of the hydrocarbon product stream from the synthesis stage and provide hydrocarbon fuel streams.

5. The plant according to claim 4, wherein said hydrocarbon fuel streams are jet-fuel and/or kerosene and/or diesel fuel and/or naphtha and/or LPG streams.

6. The plant according to claim 1, further comprising a carbon dioxide feed to the syngas generation stage.

7. The plant according to claim 1, wherein the syngas generation stage comprises a hydrogen separation stage, being arranged to receive at least a portion of the second syngas stream and provide a third syngas stream and a hydrogen-rich stream.

8. The plant according to claim 7, wherein the synthesis stage is arranged to receive at least a portion of the first or second syngas stream from the methanol cracking unit and/or at least a portion of the third syngas stream from the hydrogen separation stage.

9. The plant according to claim 1, further comprising an off-gas reforming stage, said reforming stage being arranged to receive at least a portion of the off-gas stream from the synthesis stage, and convert it to a fourth syngas stream, wherein at least a portion of said fourth syngas stream is arranged to be fed to the inlet of the synthesis stage.

10. The plant according to claim 9, wherein said off-gas reforming stage comprises at least one electrical reactor section.

11. The plant according to claim 1, further comprising an off-gas treatment stage, said off-gas treatment stage being arranged to receive at least a portion of the off-gas stream from the synthesis stage, and provide a treated off-gas, wherein at least a portion of said treated off-gas is arranged to be fed to the methanol cracking unit in the syngas generation stage.

12. The plant according to claim 1, further comprising:
a methanol synthesis stage comprising at least one methanol synthesis unit, a hydrogen feed to the methanol synthesis stage,
a carbon dioxide feed to the methanol synthesis stage,
wherein the methanol synthesis stage is arranged to convert the hydrogen feed and the carbon dioxide feed into at least a raw methanol stream and a purge stream,
a water removal stage comprising one or more distillation columns, being arranged to receive at least a portion of the raw methanol stream and to provide a methanol stream having a water content of less than 10% by weight, and a water-containing stream,
wherein at least a portion of the methanol stream from said water removal stage is provided to said syngas generation stage as the first feed of methanol.

13. The plant according to claim 12, wherein the methanol cracking unit in the syngas generation stage is arranged to receive a first portion of the methanol stream from the water removal stage, and wherein a second portion of the methanol stream is provided from the plant as a methanol product stream.

14. The plant according to claim 13, further comprising a methanol purification stage, arranged to receive the second portion of the methanol stream from the water removal stage, and provide a purified methanol product stream.

15. The plant according to claim 1, wherein the first feed of methanol, has a water content of at least 0.1%.

16. The plant according to claim 1, wherein at least one methanol cracking unit in syngas generation stage, is an electrical methanol cracking unit.

17. The plant according to claim 1, wherein the hydrogen separation stage is also arranged to provide a hydrogen-rich stream.

18. A process for providing a hydrocarbon product stream in a hydrocarbon plant according to claim 1, the process comprising the steps of:
providing the hydrocarbon plant,
supplying the first feed of methanol, having a water content of less than 10% by weight, to the syngas generation stage comprising at least one methanol cracking unit,
converting the first feed of methanol in syngas generation stage comprising at least one methanol cracking unit into at least a first syngas stream, from which unconverted methanol is optionally removed to form a second syngas stream;
optionally, supplying at least a portion of the first or second syngas stream to a hydrogen separation stage, to provide a third syngas stream and a hydrogen-rich stream;
supplying the first or second syngas stream and/or the third syngas stream to the synthesis stage from the syngas generation stage, and converting it to at least a hydrocarbon product stream and an off-gas stream.

19. The process according to claim 18, wherein the first feed of methanol has a water content of less than 5% by weight.

20. The process according to claim 18, wherein the first feed of methanol has a water content of at least 0.1% by weight.

21. The process according to claim 18, wherein the plant comprises:
a methanol synthesis stage comprising at least one methanol synthesis unit,
a hydrogen feed to the said methanol synthesis stage,
a carbon dioxide feed to the said methanol synthesis stage, and
water removal stage,
wherein said process comprises the steps of:
converting the hydrogen feed and the carbon dioxide feed in the said methanol synthesis stage into at least a raw methanol stream and a purge stream,
supplying at least a portion of the raw methanol stream from methanol synthesis stage to the water removal stage, to provide a methanol stream having a water content of less than 10% by weight, and a water-containing stream,
supplying at least a portion of the methanol stream from said water removal stage to said syngas generation stage comprising at least one methanol cracking unit as the first feed of methanol.
